(19) United States
(12) Patent Application Publication
(10) Pub. No.: US 2006/0253125 A1
(43) Pub. Date: Nov. 9, 2006

(54) Microdermabrasion method and apparatus

(76) Inventor: Roger G. Ignon, Redondo Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/370,200

(22) Filed: Mar. 7, 2006

Related U.S. Application Data
(60) Provisional application No. 60/659,393, filed on Mar. 7, 2005.

(51) Int. Cl. A61B 17/50

(52) U.S. Cl. 606/131

(57) ABSTRACT

A method and system for performing microdermabrasion on a patient's skin is provided. The method comprises providing a handpiece having a supply lumen, a return lumen, and distal cap having a window. The supply lumen and return lumen are operatively connected to a control system. A flow of crystals is introduced through the supply lumen. The flow of crystals is directed from the supply lumen at the window. The patient's skin is abraded with the crystals. The crystals are drawn in a proximal direction through the return lumen. A mode selection switch on the control system can be switched. An abrasive area is added to the handpiece. The abrasive area is positioned against a patient's skin. A suction force is applied through the return lumen to the patient's skin. The abrasive surface is moved across the patient's skin surface.

### RELATED APPLICATIONS

This application relates to and claims the benefit of the Provisional Application 60/659,393, filed Mar. 7, 2005, which is hereby incorporated by reference in its entirety.

### BACKGROUND OF THE INVENTION

1. Field of the Inventions

This invention relates generally to cosmetic apparatuses and methods used for skin treatment, and more specifically to skin abrasion treatments.

2. Description of the Related Art

Microdermabrasion is a traditional technique for treating skin conditions and defects. It involves removing, typically through abrasion, the superficial layers of the skin. Crystal microdermabrasion is a type of microdermabrasion. In crystal microdermabrasion, an air stream that carries aluminum oxide crystals is applied to the skin and the aluminum oxide crystals are used to abrade the superficial layers of the skin. In the microdermabrasion technique, the velocity and density of crystals within the stream of air is related to the degree of abrasion which can occur over a fixed period of time. In the past, the crystal velocity has been controlled primarily by providing a bleed valve for the introduction of additional air into the stream of air. Crystal microdermabrasion devices typically include handpieces that have handles and caps which define an abrasion chamber. The crystals and the stream of air are introduced into the abrasion chamber through a nozzle and along a supply path having a distal component. A return orifice, which communicates with the abrasion chamber, draws the flow of crystals along a return path having a proximal component. U.S. Pat. No. 6,673,082 is an example of such a crystal microdermabrasion device.

While crystal microdermabrasion has proven to be an effective form of microdermabrasion, other microdermabrasion techniques have also been developed. For example, U.S. Pat. No. 6,629,983 discloses a microdermabrasion device that includes a handpiece with an abrasive distal end portion. The abrasive end portion is moved over the skin while suction is applied through an opening in the end portion. The suction draws the skin against the abrasive end portion and draws away abraded skin debris. Other microdermabrasion devices utilize a moving (e.g., rotating) abrasive end portion that is moved over the skin to remove skin layers.

As with crystal microdermabrasion, these other microdermabrasion techniques have proven to be effective in treating many skin conditions and defects. However, they may not be effective in all applications or appropriate for treating all skin conditions or defects.

### SUMMARY OF THE INVENTIONS

Accordingly, one embodiment of the invention comprises a method of performing microdermabrasion on a patient's skin. The method comprises providing a handpiece having a supply lumen, a return lumen, and distal cap having a window. The supply lumen and return lumen are operatively connected to a control system. A flow of crystals is introduced through the supply lumen. The flow of crystals is directed from the supply lumen at the window. The patient's skin is abraded with the crystals. The crystals are drawn in a proximal direction through the return lumen. A mode selection switch on the control system can be switched. An abrasive area is added to the handpiece. The abrasive area is positioned against a patient's skin. A suction force is applied through the return lumen to the patient's skin. The abrasive surface is moved across the patient's skin surface.

In another embodiment, a method of treating a patient's skin is provided. The method comprises providing a microdermabrasion device capable of operating in a first mode that utilizes a crystal-laden stream to abrade a patient's skin and a second mode that applies a suction force to a patient's skin to bring the patient's skin in contact with an abrasive surface. In the first mode, a patient's skin is abraded with the crystal laden stream. The device is switched to the second mode in which the patient's skin is abraded by applying a suction force to the patient's skin to bring it in contact with the abrasive surface.

In other embodiments, a skin treatment system comprises a handpiece having a first tip. The first tip is configured to be releasably attached to the handpiece and to deliver a crystal-laden abrasive stream to a patient. A second tip has an abrasive element. A console is operatively connected to the handpiece. The console has a switch to change from a first mode in which the crystal-laden abrasive stream and a suction force is supplied to the handpiece and a second mode in which substantially only the suction force is applied to the handpiece.

In yet other embodiments, a skin treatment system comprises a vacuum source, a source of abrasive material, a handpiece operatively connected to the vacuum source and the source of abrasive material, and a valve system. The valve system is configured to operate in a first mode and a second mode. In the first mode, the valve system is configured to supply the abrasive material to the handpiece while a suction force is also supplied. In the second mode, the valve system is configured to supply a suction force to the handpiece and while preventing the flow of abrasive material to the handpiece. A switch moves the valve system from the first mode to the second mode.

These and other features and advantages of the invention will become more apparent with a description of preferred embodiments and reference to the associated drawings.

### BRIEF DESCRIPTION OF THE DRAWINGS

FIG. 1 is a perspective view of a microdermabrasion apparatus;

FIG. 2 is the schematic view of the apparatus illustrated in FIG. 1.

FIG. 3 is an exploded view of a handpiece adapted for the apparatus of FIG. 1, the handpiece is configured to releasably attach to a cap;

FIG. 4 is a front elevation view of an air control device associated with the handpiece of FIG. 3;

FIG. 5 is a cross-section view of the air control device (taken along lines 5-5 of FIG. 4.) and the associated cap for microdermabrasion;

FIG. 6a is front elevation view of an additional embodiment of the microdermabrasion handpiece cap;

FIG. 6b is a side view and axial cross-section of the cap illustrated in FIG. 6a;

FIG. 7a is a front elevation view of a further embodiment of the microdermabrasion handpiece cap;

FIG. 7b is a side view and axial cross-section of the cap illustrated in FIG. 7a;

FIG. 8 is a side elevation view of a set of endermologie massage handpieces each offering a different size in order to permit control over the magnitude of suction and the area of application;

FIG. 9 is a front elevation view of a crystal supply station and crystal return station associated with the present invention; and

FIG. 10 is a perspective view of a cap in accordance with anther embodiments.

FIG. 11 is a cross-sectional view of the air control device and the associated cap for dermabrasion.

### DETAILED DESCRIPTION OF THE PREFERRED EMBODIMENTS

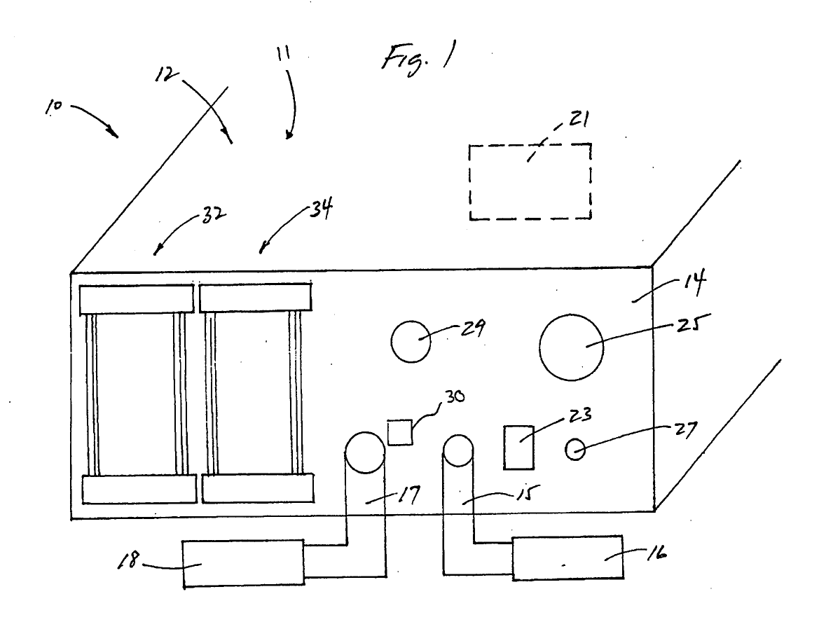

An exemplary embodiment of a microdermabrasion apparatus 10 is schematically illustrated in FIG. 1. The apparatus 10 includes a console 11 comprising single housing 12 having a face plate 14 which is adapted for coupling through a flexible hose 15 to a abrasion handpiece 16. As will be explained in more detail below, the illustrated embodiment also includes a optional second handpiece 18, which is an endermologie handpiece configured to provide therapeutic massage. However, it should be appreciated that in modified embodiments the apparatus 10 may be formed without the second handpiece, with additional handpieces and/or a handpiece configured for different purpose (e.g., light therapy, chemical therapy, etc.).

Enclosed within the single housing 12 is a source of vacuum such as a vacuum pump 21. The pump 21 is activated by a power switch 23 and coupled through a vacuum gauge 25 and bleed valve 27 to a 3-way mode switch 29. In the illustrated embodiment, the switch 29 is, in turn, coupled to the abrasion handpiece 16 and endermologie handpiece 18. As will be explained in detail below, the illustrated embodiment also includes a mode switch 30 that is configured be operated to change the operating mode of the abrasion handpiece 16. In some embodiments, the switch 30 is used to switch the handpiece 16 between crystal microdermabrasion mode and an abrasive tip mode. With continued reference to FIG. 1, a crystal supply station 32 and crystal return station 34 are disposed on the faceplate 14 of this embodiment.

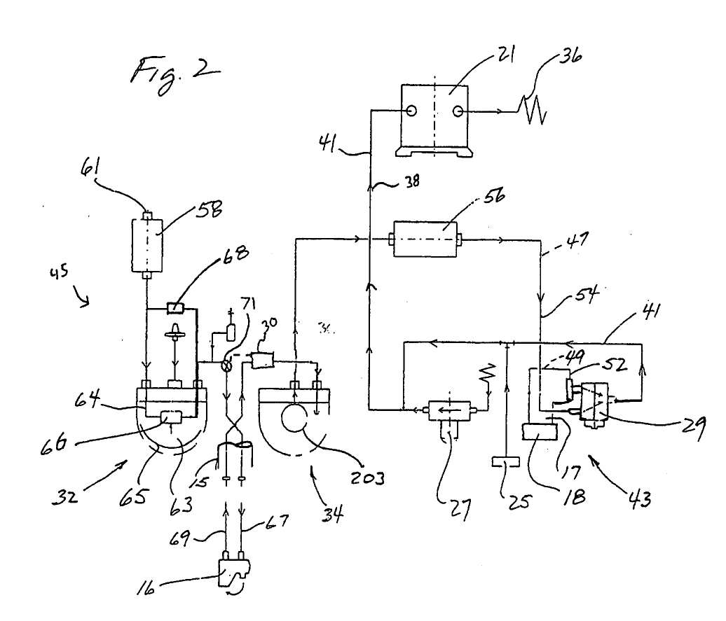

With reference now to FIG. 2, the vacuum pump 21 and provides motive power for the apparatus 10. The pump 21 has an exhaust 36 and power sufficient to pull a stream of air 38 through a primary conduit 41. The magnitude of air pressure within the conduit 41 can be controlled by the bleed valve 27 which in the preferred embodiment is a needle valve.

As illustrated, the primary conduit 41 can be coupled to the 3-way valve or mode switch 29. By operation of this switch 29, suction can be applied alternatively to an endermologie section 43 or an abrasion section 45 of the apparatus 10. Thus the switch 29 can be used to divert the stream of air 38 alternatively to form a first air stream 49 in a secondary conduit 52 in the endermologie section 43, or alternatively to form a first stream of air 47 in a secondary conduit 54 in the abrasion section 45, or alternatively a second stream of air 49 in a secondary conduit 52 in the endermologie section 43.

In the endermologie section 43 the second air stream 49 in the secondary conduit 52 provides suction at the handpiece 18. The first air stream 49 then passes back through the flexible hose 17 and into the mode switch 29 where the stream of air 38 is drawn through the primary conduit 41 by the vacuum pump 21.

Alternatively, the first switch 29 can be set to draw the first air stream 47 through the conduit 54 in the abrasion section 45. In the illustrated embodiment, the conduit 54 is in turn coupled through HEPA filters 56, the crystal return station 34, the crystal supply station 32 and a second HEPA filter 58. The filter 58 in this case provides an air inlet 61 to the abrasion section 45.

As mentioned above, it should be appreciated that in other embodiments the apparatus 10 need not include the second handpiece 18, the endermologie section 43 and/or one or more of its related components including, for example, the first switch 29. In one such embodiment, the primary conduit 41 may be directly or indirectly connected to the secondary conduit 54 in the abrasion section 45.

A supply of crystals (e.g., aluminum oxide crystals) 63 is disposed at the crystal supply station 32 where the secondary conduit 54 is connected to a pick-up tube 64 in a canister 65. In a manner discussed in greater detail below, the pick-up tube 64 can be provided with a crystal pick-up 66 which extends into the crystals 63 within the canister 65. In this manner, a flow of crystals 63 can be provided in the first air stream 47 as it is introduced through a supply lumen 67 in the flexible hose 15. The supply lumen 67 in turn introduces the flow of crystals 63 to the abrasion handpiece 16 which is adapted to be held by the surgeon or technician and applied to the skin of the patient.

In the illustrated embodiment, the crystal supply station 32 advantageously includes a bypass valve 68 which extends between the HEPA filter 58 and the supply lumen 67 of the handpiece 16. Thus, the bypass valve 68 effectively extends across the inlet and the outlet of the crystal supply station 32. When the bypass valve 68 is open, suction is applied directly to the filter 68 and a portion of the air which would otherwise be input to the crystal supply station 32 is diverted to the output of the crystal supply station 32. As a result, the flow of air in the pickup tube 64 is decreased and the volume of crystals introduced into the crystal pickup 67 is commensurately reduced. At the output of the crystal supply station 32, the bypass air is recombined with the air in the pickup tube 64 so that the velocity of air introduced to the handpiece 16 is substantially constant. However, with a decrease in the volume of crystals introduced into the pickup tube 64, the density of the crystals is reduced. Thus the bypass valve 68 provides a mechanism for varying the crystal density without significantly adjusting the crystal velocity.

The used crystals 63 can be removed from the handpiece 16 through a return lumen 69 in the flexible hose 15. This flow of crystals 63 from the handpiece 16 is directed into the crystal return station 34, which is discussed in greater detail below. The debris and used crystals are removed from the first air stream 47 at the return station 34, as the first air stream 47 is directed through the filters 56 and the conduit 54 to the 3-way valve or mode switch 29.

In the illustrated embodiment, the abrasion section 45 would be activated through the 3-way mode selection switch 29 to facilitate skin abrasion by way of the handpiece 16. At the completion of this procedure, or in a totally different procedure, the mode switch 29 could be moved to its alternate position thereby activating the endermologie section 43. However, as mentioned above, modified embodiments need not include the endermologie section 43 and/or the first switch 29.

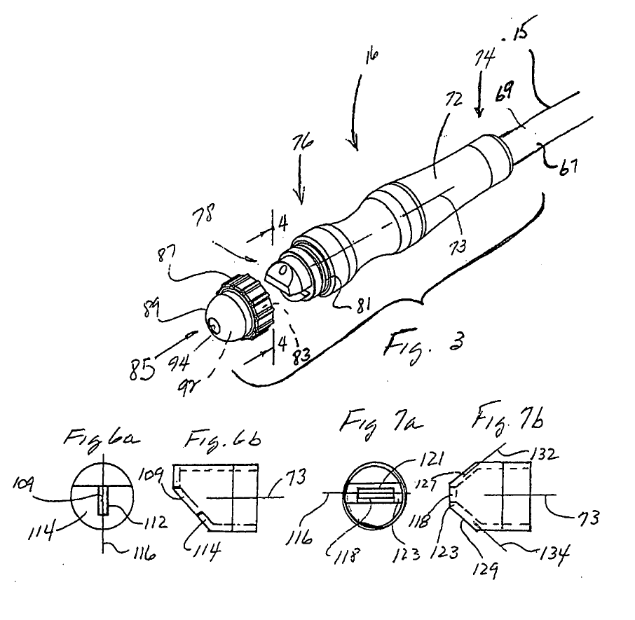

Exemplary embodiments of the abrasion handpiece 16 will now be described with reference to FIGS. 3-5. With initial reference to FIG. 3, the handpiece 16 may include a handle 72 having an axis 73 extending longitudinally between a proximal end 74 and a distal end 76. An air stream control device 78 is disposed at the distal end 76 of the handle 72 in fluid communication with the lumens 67 and 69 and the flexible hose 15. The device 78 can be provided with external threads 81 which register with internal threads 83 on a cap 85. As used herein, the terms “cap” and “tips” are used interchangeably. Alternatively the cap 85 can be friction fit onto the device 78 to facilitate a proper orientation of these two structures. The cap 85 can be provided with a knurled circumference 87 and an end wall 89 which forms with the device 78 and abrasion chamber 92. An abrasion window 94 in the end wall 89 provides access to the abrasion chamber 92.

An embodiment 86 of the air stream control device 78 is illustrated in greater detail in the front elevation view of FIG. 4 and the cross-section view of FIG. 5. From these views it can be seen that the device 78 can include a supply nozzle 101 which is disposed in fluid communication with the lumen 67 of the hose 15. The nozzle 101 receives the flow of crystals 63 from the supply lumen 67 and introduces that flow into the abrasion chamber 92. In this embodiment, the nozzle 101 is positioned to direct the flow of crystals from the lumen 67 into the window 94. In an embodiment wherein the window 94 is disposed along the axis 73, and the lumen 67 can be positioned in a parallel spaced relationship with the axis 73, the nozzle 101 is disposed at an angle a relative to the axis 73. Thus the flow of crystals 63 can be directed along a supply path 103 which has a distal component and an angle α relative to the axis 73. With the window 94 disposed at the distal most point of the end wall 89, the supply path 103 of this embodiment will always have a component in the distal direction.

After the flow of crystals 63 has abraded the patient's skin through the window 94, the used crystals are then drawn along a return path 105 to an orifice 107 in the device 78. This orifice 107 is in fluid communication with the return lumen 69 of the hose 15 which sucks the crystals 63 into the hose 15 and from the hose 15 into the return station 34.

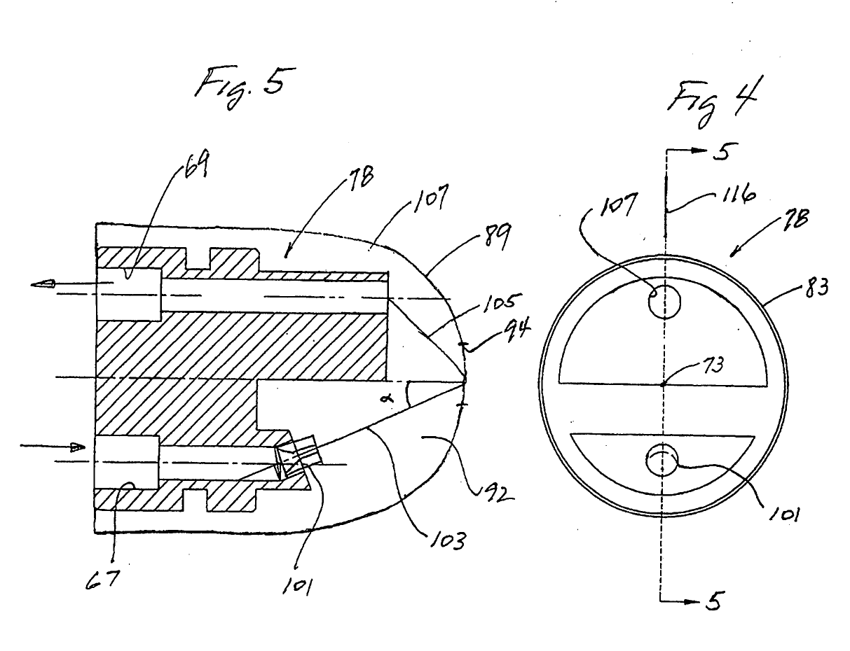

It will be noted that in the embodiment of FIG. 5, the window 94 has the configuration of a circle having its center disposed along the axis 73. Other shapes for the window 94 have also been found advantageous. In one such embodiment illustrated in FIGS. 6a and 6b, the window 94 has the configuration of a rectangle 109 having a long side 112. This window 109 is formed in an end wall 114 which has a generally planer configuration and is positioned at an angle to the axis 73. The window 109 is formed in the end wall 114 with its long side 112 disposed generally parallel to a plane 116 (FIG. 4) passing through the nozzle 101 and orifice 107.

In another embodiment illustrated in FIGS. 7a and 7b, a rectangular window 118 similar to the window 109 and having a long side 121 is disposed in an end wall 123. This end wall 123 has a generally planer configuration and is disposed generally perpendicular to the axis 73. Extending from the window 118, are opposing sidewalls 127 and 129 which extend proximally outwardly from the associated long sides of the window 118. For example, the sidewall 127 extends generally parallel to the long side 121 of the window 118. Both of the side walls 127 and 129 are disposed in respective planes 132 and 134 which are generally parallel to the long side 121 and have an angular relationship with the axis 73. In one particular embodiment, the planes 132 and 134 of the sidewalls 27 and 29, respectfully, are generally perpendicular to each other.

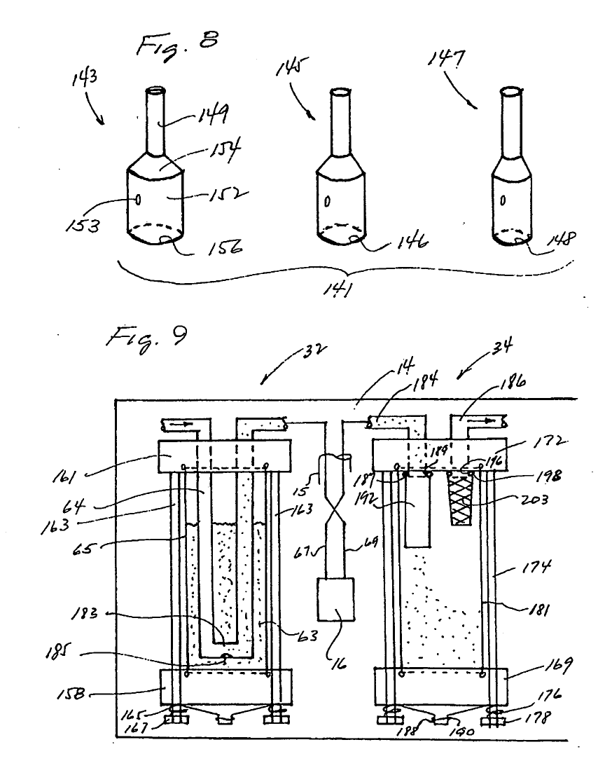

FIG. 8 illustrates a set 141 of three endermologie handpieces 143, 145, 147, which are similar to the handpiece 18 illustrated in FIG. 1. The handpiece 143 includes a handle section 149 which typically has a cylindrical configuration and a diameter which is comfortable for the surgeon or technician to hold in his hand. An operative section 152 is disposed distally of the handle section 149 and provided with a finger hole 153. This operative section 152 may also have a cylindrical configuration but will typically have a diameter greater than that of the handle section 149. A conical transition section 154 can be disposed between the handle section 149 and the operative section 152. A suction window 156 at the distal end of the handpiece 143 will typically have a diameter equivalent to that of the operative section 152.

The other handpieces 145, 147 can be similar to the handpiece 143 in that they will typically include a handle section, such as the section 149, and an operative section, such as the section 152. In the case of these handpieces 145 and 147, the diameter of the handle sections may be equivalent to the diameter of the handle section 149. However, the operative sections of the handpieces 145 and 147 will typically have windows 146 and 148, respectively, with diameters different than that of the window 156 in operative section 152. Thus, the set 141 will offer the surgeon or technician a choice of handpieces 143, 145 and 147 each having a suction window, such as the window 156, of different diameters. By selecting a particular one of the handpieces 143-147, a different suction pressure and size of operative area can be chosen.

Another advantage of the illustrated embodiment is associated with the crystal supply station 32 and crystal return station 34. With reference to FIG. 9, these stations 32 and 34 each include a bottom support 158 typically fixed to the face plate 14, and a top support 161 which is attached to posts 163 that extend through holes in the bottom support 158. Beneath the bottom support 158, the posts 163 are threaded, passed through associated springs 165, and terminated in associated nuts 167. Since the top support 161 is typically not fixed to the faceplate 14, it can be biased by the springs 165 from an extended relationship to a closely spaced relationship with the bottom support 158. This structure enables the top support 161 to be moved upwardly to the extended relationship thereby permitting insertion of the canister 65 of crystals 63. Once the canister 65 is installed, the top support 161 can be released permitting the springs 165 to bias the top support 161 into the closely spaced relationship with the bottom support 158, thereby capturing the canister 65.

The crystal return station 34 can be similarly constructed with a bottom support 169, top support 172, post 174, springs 176 and nuts 178. Operation of this structure at the crystal return station 34 can similarly permit the removable installation of a disposable canister 181.

As previously discussed with referenced FIG. 2, the first air stream can be introduced through the pick-up tube 64 which extends into the crystals 63 within the canister 65. This tube 64 will typically have a U-shaped configuration thereby permitting accommodating both ends of the tubes 64 to extend through the top support 161 while allowing an intermediate section of the tube 64 to be deeply embedded the crystal 63. The crystal pick-up 66 is preferably disposed in this intermediate section 183 near the bottom of the canister 65. In a preferred embodiment, the pick up 66 is formed as a hole in the wall of the tube 64 thereby providing access for the crystals 63 into the first air stream 47 in the tube 64. The hole 66 can be carefully sized to control the amount of crystals introduced into the tube 64 per unit volume of the first air stream 47. The larger the hole 66, the greater the amount of crystal introduced into the stream and therefore the higher the crystal density within the first air stream. The smaller the hole the less the crystal density in the first air stream. Variations in the size of the hole 66 can be provided by removable plugs or adhesive patches associated with the intermediate section 183.

The crystal return station 34 can be constructed in a manner similar to that of the crystal supply station 32 except that the input to the station 34 is provided by an inlet tube 184 in communication with the return lumen 69 from the tube 15, and an exit tube 186 in communication with secondary conduit 54 the first air stream 47. Otherwise, the bottom support 169, top support 172, post 174, springs 176 and nuts 178 can function in the manner previously discussed to permit the removable insertion of the canister 181.

The canister 181 can be provided with an O-ring 187 which defines an inlet hole 189 into the canister 181. This O-ring 187 forms a seal with the inlet tube 184, which is in fluid communication with the return lumen 69 of the handpiece 16. A downspout 192 extends from the O-ring 187 into proximity with the opposite end of the canister 181.

As the debris and used crystals 63 exit the handpiece 16, they travel along the return lumen 69 and the inlet tube 184 to the return station 32, where they pass through the downspout 192 and are collected in the canister 181. In an exit passage, clean air is provided to the secondary conduit 54 which extends through a hole 196 defined by an O-ring 198 disposed in the top of the canister 181. Attached to the O-ring 198 is a filter 203 which is preferably pleated and may be formed of paper or fabric.

The filter 203 provides filtration of the air exiting the crystal return station 34 into the conduit 54. Since this exit air forms the first air stream which in turn must pass through the 3-way mode selector valve 29 and the vacuum pump 21, it is important that the crystals 63, and any fragments thereof, be removed by this exit filter 203.

When the canister 181 is full, it can be removed by elevating the top support 172 against the bias of the springs 176 and withdrawing the canister 181 and its O-rings 187 and 196 from the associated tubes 184 and 186. The full canister 181 can then be discarded and replaced with an empty canister 181. Alternatively, the canister 181 can be made non-disposable and provided with a drain tube 188 and removable hemostat 190. This configuration will enable the contents of the canister 181 to be removed through the drain 188 and collected in a biologically hazardous bag. A similar drain and hemostat can be used with a non-disposable canister 65 in the crystal supply station 32. This configuration will enable various grit sizes to be changed through the associated drain.

Another advantage of the illustrated embodiment is the ability to provide back flushing all or various components of the system under certain circumstances. For example, if one of the crystals 63 becomes lodged in the hole 66 of the pickup tube 64, it may be desirable to blow air in a reverse direction through the crystal supply station 32. Realizing that the vacuum pump 21 will typically have an output of pressurized fluid, these and similar circumstances can be accommodated by connecting various components of the system to the output of the vacuum pump 21. The pressurized air available at this location would then be introduced into the system in a reverse direction to back flush various components. In the example noted, the output of the crystal supply station 32 could be connected to the output of the vacuum pump 21 to back flush the hole 66 and dislodge any crystals. Other components of the system which might be connected to the output of the vacuum pump 21 might include for example the conduit 41 as well as the conduits 52 and 54, the return lumen 69 of the handpiece 16, or the exit tube 186.

An advantage of the illustrated embodiment is that the abrasion section 45 and endermologie section 43 can be combined in a single unit and operated from a single vacuum source and mode selector switch. Only a single unit need be purchased by the surgeon or technician in order to perform both functions. This will be particularly appreciated in those procedures where the microdermabrasion process is facilitated by suction massage. However, as noted above, modified embodiments need not include the endermologie section 43 or may include only selected portions of this section.

Providing for pick up of the crystals 63 through the hole 66 directly into the tube 64 permits control over the crystal density with an appropriate choice of diameter for the crystal pick-up hole 185. In addition, the provision of separate valves 27 and 68 in the abrasion section 45, greatly increases the control over crystal density and velocity. Where the bleed valve 27 controls crystal velocity but not crystal density, the bypass valve 68 controls crystal density but not crystal velocity.

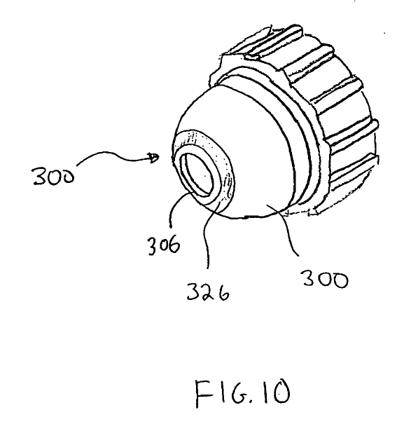

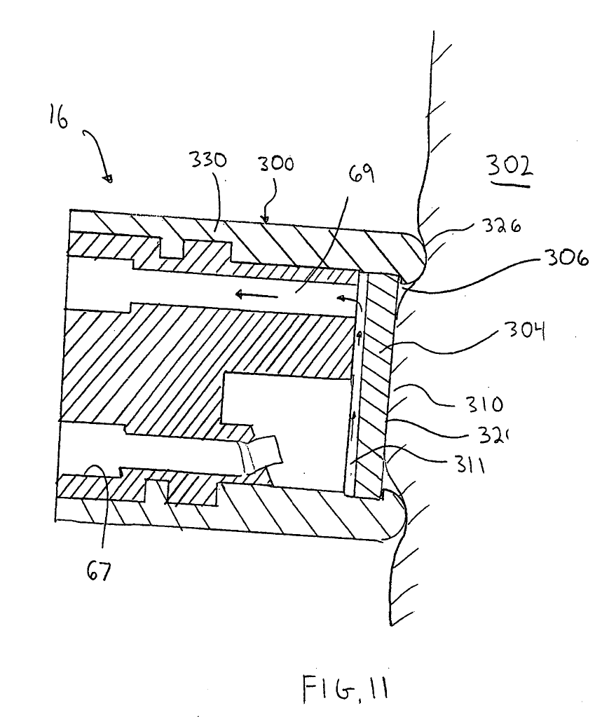

Another advantage of the illustrated embodiment is that one or more treatments can be preformed with a single handpiece. For example, in one embodiment, to perform a desired treatment, a clinician can attach different caps designed for different treatments. For example, a treatment process may comprise having the handpiece 16 operate in a plurality of modes, wherein a specially designed cap is attached, preferably removably attached, to the handpiece 16 during each mode. In some embodiments, the handpiece 16 has a cap (e.g., the cap 85) to configured for performing crystal microdermabrasion. The microdermabrasion cap 85 can be removed from the handpiece 16, and an abrasive cap 300 (see FIG. 10) can then be attached to the handpiece 16 to perform microdermabrasion without the use of a crystals as explained above. In another embodiment, the same cap may be used for different modes. For example, an abrasive cap 300 may be used in combination with a crystal laden air stream and then may be used without a crystal laden air stream. In another embodiment, the apparatus 10 may include a first handpiece 16 configured for crystal microdermabrasion and a second handpiece 18 as shown in FIG. 1 that is configured with an abrasive tip as described below.

With reference again to FIGS. 1 and 2, the mode switch 30 can be used to change the operating mode of the handpiece 16, preferably between a crystal microdermabrasion mode and abrasive tip mode. The switch 30 is used to control the fluid flow through the lumens 67 and 69. The switch 30 can be operated such that in a first or crystal mode crystals are supplied to the handpiece through the supply lumen 67 and in a second or crystal-free mode substantially only suction is provided to the handpiece 16 through the suction lumen 69. In the crystal-free mode, an abrasive cap or tip is attached to the handpiece 16 while in the crystal mode a non-abrasive tip can be attached to the handpiece 16.

As shown in FIG. 2, the switch 30 can be in communication with a valve system 71, which can inhibit or prevent the flow through the fluid lumen 67. When the valve system 71 is closed, the pump 21 can provide suction through the lumen 69. Thus, the fluid may flow through the lumen 69 while fluid does not flow through the lumen 67. Of course in modified embodiments, the valve system 71 can be located at other positions in the flow paths of the apparatus. For example, the valve 71 can be located along the lumen 67 and within the handpiece 16. The switch 30 can also be located on the handpiece 16 so that the clinician can conveniently switch operating modes of the apparatus 10.

As shown in FIG. 2, in the crystal-free mode, the flow from lumen 69 preferably still passes through the filter 203, which provides filtration of the air before entering the conduit 54. In this manner, skin debris and fragments may be removed by this exit filter 203 before reaching the vacuum 21. As mentioned above, in modified embodiments, the endermolugie section 43 or portions thereof may be omitted from the apparatus 10. In one such embodiment, the conduit 54 may be connected directly or through an intermediate component to the primary conduit 41.

FIG. 10 illustrates an abrasive cap 300 for performing dermabrasion. The dermabrasion cap 300 is similar to the caps described above, except as further described below. The cap 300 has an abrasive area 326. The surface 326 can be configured in any of a variety of manners for abrading, cutting or otherwise removing layers of skin from the patients. Fro example, the abrasive area may comprise a roughened, knurled, grooved or serrated surface configured to remove skin layers. In other embodiments, the abrasive area 326 may be formed by embedding, attaching or coating the cap with an abrasive particulate (e.g., diamond grit or other hard particulate) and/or the like. The abrasive area 326 may cover the entire portion of the cap 300 intended to contact the skin or only portions thereof.

In use, the abrasive cap 300 can be attached to the handpiece 16 (see FIG. 3). The handpiece 16 is then grasped by the practioner and manipulated until the distal end of the cap is 300 is placed against the skin. A vacuum can be applied by a pump (e.g., the pump 21 of FIG. 2) to the lumen 69, preferably while the lumen 67 does not permit fluid flow therethrough. The vacuum provides suction around an opening 306 in the cap 300. When the opening 306 in the handpiece 16 is brought into proximity with the skin 302, a portion of the skin is drawn through the opening 306 and into abrasive contact with the abrasive area 326. It should be appreciated that in other embodiments the abrasive cap 300 may have other shapes or forms. For example, the cap may have flat interface surface configured to contact the skin. The cap may also may have one or more openings 306 positioned within the abrasive area 326 or outside the abrasive area 326. In some non-limiting exemplary embodiments which use diamond grit, the surface 326 can have various grit ratings, such as 400 grit, 300 grit, 270 grit, 230 grit, 100 grit, 170 grit, and ranges encompassing such grit ratings.

In operation, the cap 300 can be used to perform dermabrasion. The cap 300 can be removably attached to the handpiece 16 so that the cap 300 can be conveniently removed in order to attach a cap suitable for crystal microdermabrasion to the handpiece 16. The caps of the handpiece 16 can be switch as desired. Any suitable means can be employed to attach the caps to the handpiece 16. For example, the caps can be threadably attached to the handpiece 16. In other embodiments, the caps can be snapped onto the handpiece 16. In still other embodiments, an abrasive element comprising an abrasive area 326 may be attached to cap suitable from crystal microdermabrasion. In yet another embodiment, the same cap may be used for crystal-free and crystal microdermabrasion.

FIG. 11 illustrates a modified embodiment in which the cap 300 is used in combination with an abrasion element 304 that is positioned within the opening 306 of the cap 300. The element 304 may be in the form of a disk that generally covers the opening 306. The vacuum provides suction around and/or through the abrasive disk or element 304 to an opening 306 in the cap 300. When the opening 306 in the handpiece 16 is brought into proximity with the skin 302, a portion of the skin 302 is drawn through the opening 306 and into abrasive contact with the abrasive element 304. In some embodiments, abrasion occurs with respect to the skin portion 310 which is drawn through the opening 306. This insures that any debris resulting from the abrasion is exposed to the vacuum and drawn off into the channel 311 and the lumen 69. The abrasion occurs not simply due to contact between the skin portion 304 and the abrasive element 304, but due to the relevant movement of the skin portion 310 and the element 304 as the handpiece 16 is moved over the skin 302. Thus, progressive portions of the skin are drawn into abrasive contact with the element 304 as the handpiece 16 is moved relative to the surface or skin 302. In this embodiment, the outer area 326 may be smooth or configured for skin removal as described above. In one embodiment, the abrasive element may comprise a porous pad with an abrasive outer layer 320. In such an embodiment, the element may be disposable or otherwise configured for limited or single use. The element 304 can be disposable so that one or more elements 304 are used in combination with a single cap. Thus, the elements 204 can be removable from the outer housing 330 of the cap 300.

As mentioned above, the housing 330 may comprise an engagement surface 326 of cap 300 can be substantially smooth or have a surface treatment for enhancing skin abrasion. For example, the surface 326 can be a smooth polished surface that can slide easily along the skin 302. Thus, the element 304 can abrade the skin 302 while the surface 326 slides smoothly along the skin. Of course, the cap can be used without an abrasive element 304 as described above with reference to FIG. 10.

Other features, systems, devices, materials, and methods and techniques that may be used in combination or sub-combinations with the embodiments disclosed herein are described in U.S. Pat. Nos. 6,673,082, 6,641,591, 6,629,983, 6,387,103, 6,299,620 6,241,739 and U.S. application Ser. No. 10/315,478 (U.S. Publication No. 2003-0167032 A1), the entirety of which is hereby incorporated by reference herein and made a part of this specification

Furthermore, the skilled artisan will recognize the interchangeability of various features from different embodiments disclosed herein. Similarly, the various features and steps discussed above, as well as other known equivalents for each such feature or step, can be mixed and matched into various combinations and sub-combinations by one of ordinary skill in this art to perform methods in accordance with principles described herein.

Additionally, the methods which is described and illustrated herein is not limited to the exact sequence of acts described, nor is it necessarily limited to the practice of all of the acts set forth. Other sequences of events or acts, or less than all of the events, or simultaneous occurrence of the events, may be utilized in practicing the embodiments of the invention.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof.

What is claimed is:

1. A method of performing microdermabrasion on a patient's skin, comprising:
providing a handpiece having a supply lumen, return lumen, and distal cap having a window, the supply lumen and return lumen operatively connected to a control system;
introducing a flow of crystals through the supply lumen;
directing the flow of crystals from the supply lumen at the window such that the patient's skin is abraded with the crystals;
drawing the crystals in a proximal direction through the return lumen;
switching a mode selection switch on the control system;
adding an abrasive area to the handpiece;
positioning the abrasive area against a patient's skin;
applying a suction force through the return lumen to the patient's skin; and
moving the abrasive surface across the patient's skin surface so as to effectively remove skin.

2. The method of claim 1, wherein adding an abrasive area to the handpiece comprises removing the distal cap and attaching to the handpiece a second distal cap that includes the abrasive area.

3. The method of claim 1, wherein adding an abrasive area to the handpiece comprises positioning an abrasive element between the window and the return lumen.

4. A method of treating a patient's skin, comprising:
providing a microdermabrasion device capable of operating in a first mode that utilizes a crystal-laden stream to abrade a patient's skin and a second mode configured to apply a suction force to a patient's skin to bring the skin in contact with an abrasive surface;
in the first mode, abrading a patient's skin with the crystal laden stream;
switching the device to the second mode; and
in the second mode, abrading a patient's skin by applying a suction force to the patient's skin to bring it in contact with the abrasive surface.

5. A skin treatment system comprising:
a handpiece;
a first tip configured to be releasably attached to the handpiece and to deliver a crystal-laden abrasive stream to a patient;
a second tip having an abrasive element; and
a console operatively connected to the handpiece, the console having a switch to change from a first mode in which the crystal-laden abrasive stream and a suction force is supplied to the handpiece and a second mode in which substantially only the suction force is applied to the handpiece.

6. A skin treatment system comprising:
a vacuum source;
a source of abrasive material;
a handpiece operatively connected to the vacuum source and the source of abrasive material;
a valve system configured to operate in a first mode and a second mode, in the first mode, the valve system is configured to supply the abrasive material to the handpiece while a suction force is also supplied, in the second mode, the valve system is configured to supply a suction force to the handpiece and while preventing the flow of abrasive material to the handpiece; and
a switch for moving the valve system from the first mode to the second mode.
